(19) United States
(12) Patent Application Publication
(10) Pub. No.: US 2023/0397977 A1
(43) Pub. Date: Dec. 14, 2023

(54) VAGINAL GRAFT DEVICES AND MEDICAL PROCEDURES

(71) Applicant: Nathan Guerette, Midlothian, VA (US)

(72) Inventor: Nathan Guerette, Midlothian, VA (US)

(21) Appl. No.: 18/207,744

(22) Filed: Jun. 9, 2023

Related U.S. Application Data
(60) Provisional application No. 63/351,000, filed on Jun. 10, 2022.

(51) Int. Cl. A61F 2/00; A61B 17/04

(52) U.S. Cl. CPC A61F 2/0045; A61B 17/0401; A61B 2018/00547

(57) ABSTRACT

The present invention includes a biological vaginal graft with a central body and a plurality of extension members. The central body has a tapered dimension from a bottom portion to a bottom portion. The extension members extend outward from the central body, disposed on opposing sides of the bottom portion. The central body and the extension members are composed of tissue graft material. The extension members, as being composed of the tissue graft material, as affixable via a grafting medical procedure to a vaginal canal of a patient.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### RELATED APPLICATIONS

The present application relates to and claims priority to U.S. Provisional Patent Application Ser. No. 63/351,000 entitled “VAGINAL GRAFT DEVICES AND MEDICAL PROCEDURES” having a filing date of Jun. 10, 2022, the entirety of which is hereby incorporated by reference.

### BACKGROUND

Problems exist seeking solutions for treating pelvic organ prolapse, urinary incontinence, fecal incontinence, urethral diverticulum, fistula, neo-vagina, and wound healing abnormalities.

Naturally occurring extracellular matrices (ECMs) have been used for tissue repair and regeneration, including usable for repair, support and stabilization of anatomical defects. There are known techniques for harvesting and preparing ECMs ahead of surgery, including sterilization and related techniques for minimizing post-surgical complications.

One common problem arises due to the nature of these grafts having a general non-defined shape. For certain types of surgeries, the graft must be cut into a specific shape or design. Because of sterilization requirements for surgical theaters, having to cut the graft during surgery adds additional complications, including causing the surgery to become longer in duration, lack of precision with available tools in an operating room, lack of knowledge by the surgeon and/or staff to create an appropriate design, as well as adding contamination concerns.

Therefore, there exists a need for a vaginal graft providing for improving not only the patient outcome but also improving the safety and timing of any attendant surgeries.

### BRIEF DESCRIPTION

The present invention includes a biological vaginal graft with a central body and a plurality of extension members. The central body has a tapered dimension from a bottom portion to a top portion. The extension members extend outward from the central body, disposed on opposing sides of the bottom portion. The central body and the extension members are composed of tissue graft material. The extension members, as being composed of the tissue graft material, are affixable via a grafting medical procedure to a vaginal canal of a patient.

Based on the tapered dimensions, the width of the top portion is smaller than the width of the bottom portion. In one embodiment, the top width is within the range of 3.5 centimeters to 4.5 centimeters and the bottom width is within the range of 5.5 centimeters to 6.5 centimeters.

In one embodiment, the extension members each have a length between 3.5 centimeters and 6.5 centimeters, extending outward from the central body. In one embodiment, the extension members each have a width ranging from 0.35 centimeters to 1.5 centimeters.

In another embodiment, the biological vaginal graft can include one or more apical arms extending outward and/or sideways from the top portion. The apical arms are composed of biological grafting material similar or identical to the central body, the apical arms are affixable to the vaginal canal of the patient. In one embodiment, the apical arms can include a length within the range of 3.5 centimeters to 6.5 centimeters and a width within the range of 0.35 centimeters to 1.5 centimeters.

In varying embodiments, the biological vaginal graft is composed of different biologically-compatible material. One embodiment includes composition of decellularized dermal allograft. Another embodiment includes composition of animal-based origin. Additional embodiments include composition of other human tissue such as fascia lata, amnion, other dermal tissues or other biologically compatible human tissues.

### BRIEF DESCRIPTION OF THE DRAWINGS

The present invention is additionally noted in the Figures as part hereof, including:

FIG. 1 illustrates one embodiment of a vaginal graft;

FIG. 2 illustrates another embodiment of the vaginal graft of FIG. 1;

FIG. 3 illustrate one embodiment of a vaginal graft;

FIG. 4 illustrates another embodiment of the vaginal graft of FIG. 3;

FIG. 5 illustrates a flowchart of the steps of one embodiment of a surgical procedure for implantation of the vaginal graft;

FIG. 6 illustrates a flowchart of the steps of one embodiment of a surgical procedure for implantation of the vaginal graft;

FIG. 7 illustrates one embodiment of an incontinence sling; and

FIG. 8 illustrates one embodiment of a collagen/biologic Y graft.

### DETAILED DESCRIPTION

The below designs and shapes, including designated measurements, describe specific implementations. The technical designs and implantation techniques can vary based on in shapes and measurements within standard deviations and the shapes and measurements noted herein are not expressly limiting but a general guideline and delineation of the technology.

Specific designs: These designs and implantation techniques address common female pelvic surgical issues. The exact dimensions and implantation technique can vary, and the specific descriptions are not limited to dimension or utility. The dimensions noted herein and as included in the attached figures are one embodiment and not exclusive or limiting values or ranges, where it is recognized by a skilled artisan that other sizes and/or ranges are within the present disclosure.

The grafts as noted herein can include any suitable graft material as recognized by one skilled in the art. In one embodiment, the graft material can be a decellularized dermal allograft having dimensions as noted herein. Moreover, the graft thickness can vary or be uniform, including but not limited to thicknesses ranging from 0.5 mm to 4 mm. The graft can either be solid or meshed or consist of other varying patterns.

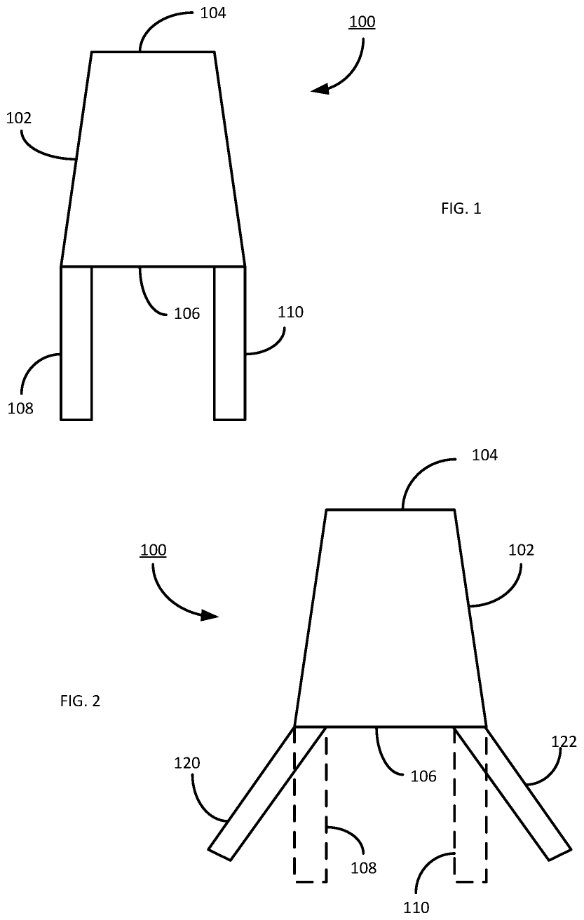

FIG. 1 illustrates one embodiment of a posterior vaginal graft 100. The graft 100 includes a central body 102 having a top portion 104 and a bottom portion 106. In this embodiment, the vaginal graft 100 further includes extension members 108, 110.

The vaginal graft 100 includes a defined shape providing for implantation within a patient. In this embodiment, the shape includes a tapered dimension of the body 102 such that the top portion 104 has a smaller in width than the bottom portion 106, providing a trapezoidal shape.

The extension members 108 and 110 extend outward from the central body 102 and are attached at the top portion 104. In this embodiment, the members 108 and 110 extend directly outward in a 90 degree angle.

In the FIG. 1 embodiment, the top portion 104 and the bottom portion 106 are illustrated as having a linear or flat surface, lacking any curvature. In varying embodiment, the shape of the top portion 104 and the bottom portion 106 can vary and the shape of these portions are not expressly limited to a flat or linear surface. For example, the top portion 104 and/or bottom portion 106 can have a arcuate or angular shape.

In one embodiment, the top portion 104 can have a length within a range of 3.5 centimeters to 4.5 centimeters. In one embodiment, the bottom portion 106 can have a length within a range of 5.5 centimeters to 6.5 centimeters.

While FIG. 1 illustrates the members 108, 110 extending outward at a right angle from the central body 102, the members 108 and 110 can extend out at any suitable angle. For example, in one embodiment the members 108, 110 can extend outward at an angle in a range of up to 60 degrees outward from the bottom portion 106 and at an angle in a range of up to 30 degrees inward from the bottom portion 106.

In one embodiment, the members 108, 110 can have a length within a range of 3.5 centimeters to 6.5 centimeters, extending outward from the bottom portion. In a further embodiment, the members 108, 110 may include a width within the range of 0.35 centimeters to 1.5 centimeters.

For further clarification, FIG. 2 illustrates another embodiment of the graft 100 include the body 102 with top portion 104 and bottom portion 106. Illustrated in dashed lines are the members 108, 110 from FIG. 1. By contrast, this embodiment provides for arms 120 and 122 extending angularly outward from the body and directly downward from the bottom portion 106. The arms 120 and 122 can extend outward from the base portion 106 at any suitable angle.

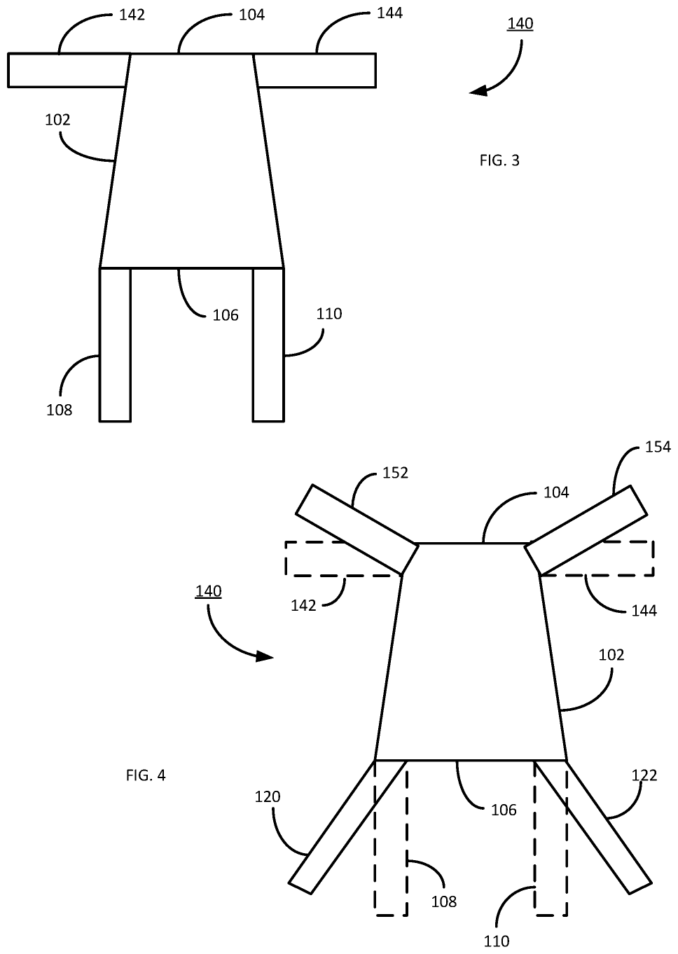

FIG. 3 illustrates another embodiment of a vaginal graft 140. This embodiment, similar to the graft 100, includes the body 102, top portion 104, and bottom portion 106. This embodiment includes members 108 and 110 extending from the bottom portion 106. Further attached to the body 102, apical arms 142 and 144 extend outward from the top portion 104. Illustrated in FIG. 3, the arms 142 and 144 extend directly outward from top portion 104 running in alignment with the top portion 104. In this embodiment, the arms 142, 144 connect to the top portion of the body 102.

As noted above regarding FIG. 1, the top portion 104 (and bottom portion 106) are not expressly limited to be linear in shape, but can include curvatures. Similarly the apical arms 142 and 144 are not expressly limited to being parallel with the top portion 104.

In one embodiment, the apical arms 142, 144 may include a length within the range of 3.5 centimeters to 6.5 centimeters. In a further embodiment, the arms 142, 144 may include a width within the range of 0.35 centimeters to 1.5 centimeters.

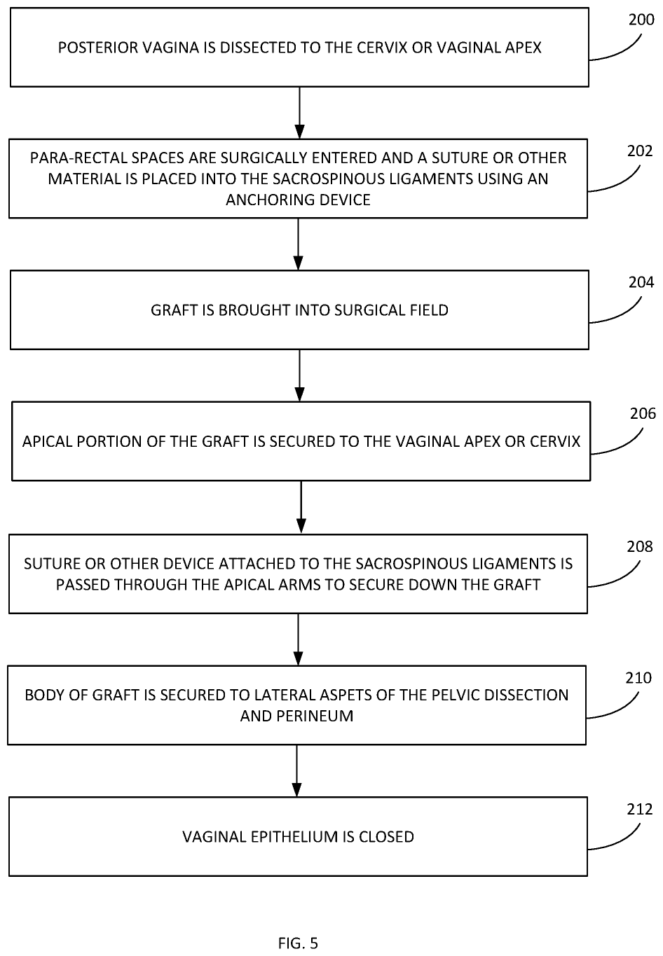

FIG. 4 illustrates an alternative embodiment of the vaginal graft 140 of FIG. 3. The graft 140 includes the body 102, top portion 104, bottom portion 106, members 108, 110. In lieu of the arms 142, 144 illustrated in dashed lines, this embodiment includes are 152, 154 angled outward from the top portion 104 at an angle relative to the body 102.

The arms 152, 154 can extend outward from any suitable angle. In one embodiment, where there is a curvature of the top portion 104, the arms 152, 154 can continue the curvature in extending outward (and/or upward) from the top portion 104.

Further visible, this embodiment of FIG. 4 can include the members 120, 122 extendable in varying angles outward from the bottom portion 106.

The grafts 100 and 140, including varying embodiments noted herein, can be composed of any suitable material providing for implantation within a patient. For example, in one embodiment the tissue graft material of the vaginal graft is composed of decellularized dermal allograft. For example, in one embodiment the tissue graft material of the vaginal graft is composed of animal-based original.

The grafts 100 and 140 include the noted elements and associated dimensions and angles of the elements for providing implantation within a patient during surgery. The prior techniques required manipulation or cutting of allograft material during surgery, creating numerous safety an efficacy concerns. The grafts 100 and 140 and associated elements are predesigned for surgical implantation without requiring modification of the graft material within the surgical theater.

Where additional embodiments of vaginal grafts are not below, FIG. 5 provides a flowchart of the steps of one embodiment of an implantation method for a vaginal graft. The method is a surgical procedure performed within a sterile surgical field, e.g. operating room.

The method includes a first step, step 200, of dissecting the posterior vagina to either the cervix or vaginal apex. The degree of dissection can be based on a variety of factors including the repair field of the patient.

In step 202, the para-rectal spaces are entered. Furthermore in step 202, an anchoring device is used to place suture or other material into the sacrospinous ligaments. This suture or other material placement can be performed using known placement techniques with an anchoring device, as recognized by a skilled artisan.

In step 204, the graft is brought into the surgical field. The graft is the graft 100 of FIGS. 1-2 above. The graft having the defined shape can be brought into the surgical field without requiring modification of the graft prior to implantation. Therein using the graft 100 provides for safer and quicker surgical procedure for the patient being operated on, but also eliminating user error and poor manufacturing associated with the prior techniques of manual cutting of the graft just prior to implantation, as well as suboptimal design or cut shapes by inexperienced surgeons and/or staff.

In step 206, the apical portion of the graft is secured to the vaginal apex or cervix. In the embodiment using the vaginal graft 100, the apical portion includes the members 108, 110. Step 206 can be accomplished using either semi-permanent or absorbable suture or similar devices with multiple attachments into the vaginal apex and/or cervix uteri. The number of attachments can vary, as recognized by a skilled artisan.

Once secured, step 208 includes the suture or other device attached to the sacrospinous ligaments is passed through the apical arms in the appropriate locations and secured down.

In step 210, the body of the graft is secured to the lateral aspects of the pelvic dissection and perineum. The lateral aspect of the mid-portion of the graft is secured to the endopelvic fascia overlaying the iliococcygeus muscle at the level of the mid-vagina. The distal tail of the graft is then appropriately trimmed, if needed, to match the anatomy of the patient and anchored to the perineal body with multiple absorbable sutures or similar material.

Additional surgical repairs are performed as needed to the patient.

Step 212, the vaginal epithelium is closed using any suitable closure technique, including for example stitches.

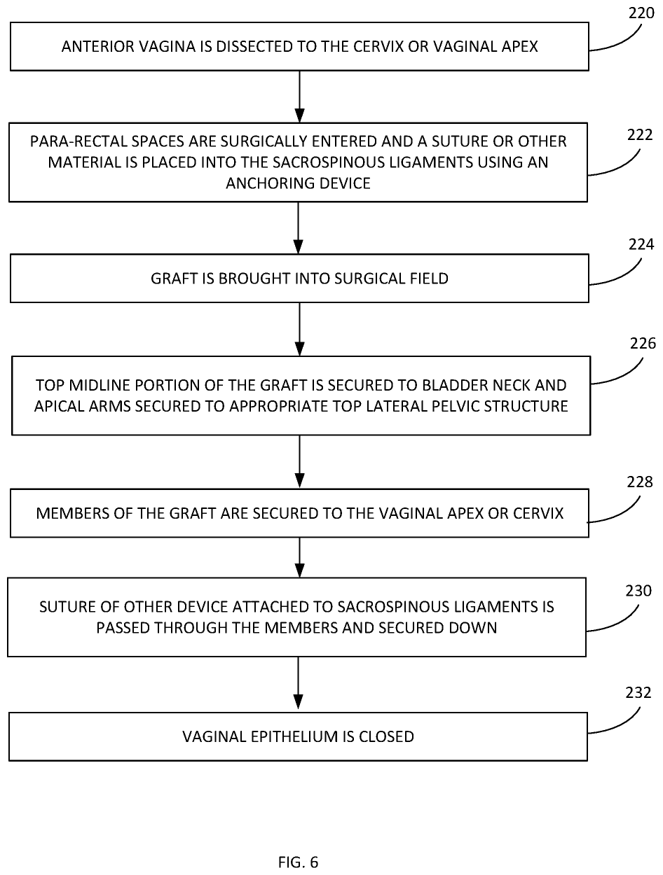

FIG. 6 illustrates a flowchart of the steps of a surgical method for an implantation technique for the anterior vaginal graft. Step 220 includes the anterior vagina is dissected to the cervix or vaginal apex.

In step 222, the para-rectal spaces are entered and an anchoring device is used to place suture or other material into the sacrospinous ligaments.

In step 224, the graft is brought into the surgical field.

In step 226, the top midline portion of the graft is secured to bladder neck and the lateral top arms are secured to the Arcus Tendinous Fascia Pelvis using absorbable sutures, anchors or other similar techniques.

In step 228, the apical body of the graft is secured to the vaginal apex or cervix, for example members 108, 110 of FIGS. 1-4. In one embodiment, securing of the graft can be performed in similar or identical fashion to attachment techniques noted in steps 206, 208 of FIG. 5.

In step 230, the suture or other device attached to the sacrospinous ligaments is passed through the apical arms in the appropriate locations and secured down.

Additional repairs are performed as needed. The vaginal epithelium is closed, step 242.

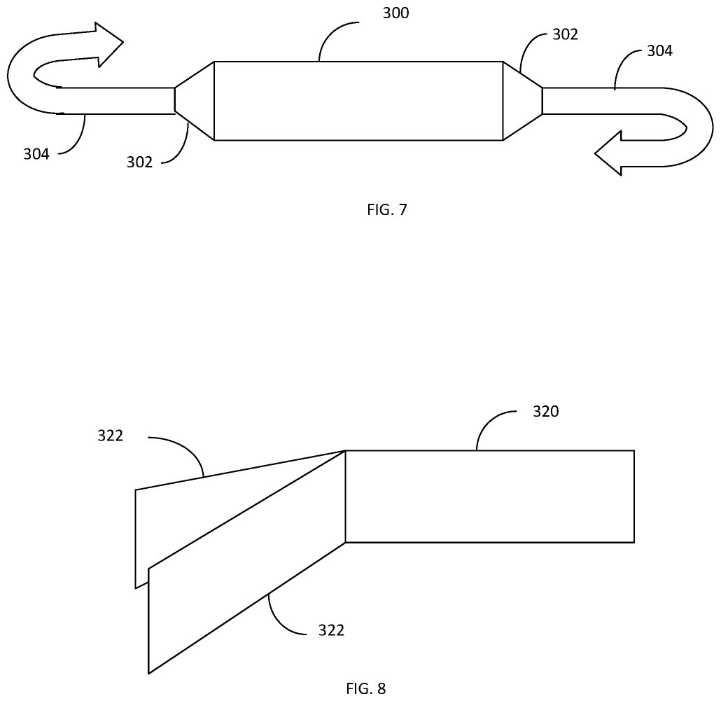

Another embodiment includes an incontinence sling, as illustrated in FIG. 7. In this embodiment, the sling is composed of a graft with a narrow rectangular body 300 and tapered ends 302. The graft can have pre-attached suture with or without anchors to accommodate a variety of implantation techniques.

In one embodiment, the body 300 can have a dimension of 1 cm by 8 cm or within a range of 0.5 cm-1.5 cm by 6 cm-10 cm. The tapered ends 302 are connectable to suture, anchors, or other suitable elements 304 for implantation.

Common implantation techniques will be described below for this design, by way of example, but the design is not limited to these implantation techniques.

One embodiment includes an implantation technique for the incontinence sling of FIG. 7. In one embodiment, the implantation can include a single-incision: The vaginal epithelium is incised over the mid-urethra and dissected to the lateral vaginal sulcus or urogenital diaphragm. The sling with attached anchoring device is introduced into the surgical field and the anchors are placed into the obturator membrane or in retropubic fashion to appropriately tension the sling below the urethra. The vaginal epithelium is closed.

In another embodiment, implantation can be via transobturator: The vaginal epithelium is incised over the mid-urethra and dissected to the lateral vaginal sulcus. A needle or other device is passed through a skin incision superficial to the obturator membrane. The sling is introduced into the surgical field and the needle is used to bring top portions of the sling out of the skin incision and the sling is appropriately tensioned below the urethra. The vaginal epithelium is closed.

In another embodiment, implantation can be via retropubic full length: The vaginal epithelium is incised over the mid-urethra and dissected to the urogenital diaphragm. 2 skin incisions are created superior and lateral to the midline of the pubic symphysis. A needle or other device is used to pass the sling through the urogenital diaphragm and ipsilateral skin incision on both sides and the sling is appropriately tensioned below the urethra. The excess sling material is removed and the Incisions are closed.

In another embodiment, implantation can be via retropubic non-anchored sling: The vaginal epithelium is incised over the mid-urethra and dissected to the urogenital diaphragm. A transverse skin incision is created superior to the midline of the pubic symphysis. A needle or other device is used to pass the sling through the urogenital diaphragm and ipsilateral skin incision on both sides of the vaginal incision and the sling is appropriately tensioned below the urethra by tying attached suture or other materials together in the midline. The Incisions are closed.

FIG. 8 illustrates another embodiment, a collagen/biologic “Y” graft. The collagen/biologic “Y” graft is composed of three arms (2 top arms 322 and an apical arm 320). This graft is designed but not limited to use for abdominal approach repair of pelvic organ prolapse. The “Y” graft can be meshed or solid in design and can have cured, straight, or angled edges.

In one embodiment, the arms 322 and 320 can each have a dimension within a range of 8 cm to 12 cm in length and a width of 3 cm to 5 cm.

In one embodiment, the Collagen/biologic “Y” graft includes a plurality of Implantation Techniques.

A first implantation technique is appropriate access and dissection is accomplished in a standard fashion. The top arms of the Y-graft are appropriately measured and the graft is introduced into the surgical field. The top arms are secured to the anterior and posterior vagina using a self-anchoring suture in a continuous sewing technique. The vagina is appropriately supported and the apical portion of the graft is secured to the anterior longitudinal ligament over the sacrum in a standard fashion. Additional repairs are performed as needed and the incisions are closed.

A second implantation technique is appropriate access and dissection is accomplished in a standard fashion. The top arms of the Y-graft are appropriately measured and the graft is introduced into the surgical field. The top arms are secured to the anterior and posterior vagina using interrupted suture in appropriate locations on the anterior and posterior top arms. The vagina is appropriately supported and the apical portion of the graft is secured to the anterior longitudinal ligament over the sacrum in a standard fashion. Additional repairs are performed as needed and the incisions are closed.

Notably, the examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Further, the present invention encompasses present and future known equivalents to the known components referred to herein by way of illustration.

The foregoing description of the specific embodiments so fully reveals the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein.

What is claimed is:

1. A biological vaginal graft comprising:
a central body with a top portion and a bottom portion, the central body having a trapezoidal dimension; and
a plurality of extension members extending outward from the bottom portion, the plurality of extension members affixable via a grafting medical procedure to a vaginal canal of a patient;
wherein the plurality of extension members are disposed on opposing edges of the bottom portion;
wherein the central body and the plurality of extension members of the biological vaginal graft are composed of tissue graft material.

2. The biological vaginal graft of claim 1, wherein a top width of the top portion is less than a bottom width of the bottom portion.

3. The biological vaginal graft of claim 2, wherein the top width is within the range of 3.5 centimeters to 4.5 centimeters and the bottom width is within the range of 5.5 centimeters to 6.5 centimeters.

4. The biological vaginal graft of claim 1, wherein each of the plurality of extension members has a length within a range of 3.5 centimeters to 6.5 centimeters extending outward from the bottom portion.

5. The biological vaginal graft of claim 1, wherein each of the plurality of extension members has a width within a range of 0.35 centimeters to 1.5 centimeters.

6. The biological vaginal graft of claim 1 further comprising:
a plurality of apical arms extending outward from the top portion, the plurality of apical arms affixable via the grafting medical procedure to the vaginal canal of a patient.

7. The biological vaginal graft of claim 6, wherein each of the apical arms has a length within the range of 3.5 to 6.5 centimeters and width within the range of 0.35 centimeters to 1.5 centimeters.

8. The biological vaginal graft of claim 1, wherein the tissue graft material is composed of decellularized dermal allograft.

9. The biological vaginal graft of claim 1, wherein the tissue graft material is composed of animal-based origin.

10. The biological vaginal graft of claim 1 available for surgical attachment to the patient using the grafting medical procedure based on the existing central body and the plurality of extension members without requiring modification of the vaginal graft within a surgical theater.

11. A biological vaginal graft comprising:
a central body with a top portion and a bottom portion, the central body having a trapezoidal dimension, the top portion having a top width is within a range of 3.5 centimeters to 4.5 centimeters and the bottom portion having a bottom width within a range of 5.5 centimeters to 6.5 centimeters; and
a plurality of extension members extending outward from the bottom portion, the plurality of extension members affixable via a grafting medical procedure to a vaginal canal of a patient;
wherein the plurality of extension members are disposed on opposing edges of the bottom portion, each of the plurality of extension members has a length within a range of 3.5 centimeters to 6.5 centimeters extending outward from the bottom portion;
wherein the central body and the plurality of extension members of the biological vaginal graft are composed of tissue graft material.

12. The biological vaginal graft of claim 11, wherein the tissue graft material is composed of decellularized dermal allograft.

13. The biological vaginal graft of claim 11, wherein the tissue graft material is composed of animal-based origin.

14. The biological vaginal graft of claim 11 available for surgical attachment to the patient using the grafting medical procedure based on the existing central body and the plurality of extension members without requiring modification of the vaginal graft within a surgical theater.

15. A biological vaginal graft comprising:
a central body with a top portion and a bottom portion, the central body having a trapezoidal dimension;
a plurality of extension members extending outward from the bottom portion, the plurality of extension members affixable via a grafting medical procedure to a vaginal canal of a patient; and
a plurality of apical arms extending outward from the top portion, wherein each of the apical arms has a length within the range of 3.5 to 6.5 centimeters and width within the range of 0.35 centimeters to 1.5 centimeters, the plurality of apical arms affixable via the grafting medical procedure to the vaginal canal of the patient;
wherein the plurality of extension members are disposed on opposing edges of the bottom portion;
wherein the central body and the plurality of extension members of the biological vaginal graft are composed of tissue graft material.

16. The biological vaginal graft of claim 15 further comprising:
a top width of the top portion is less than a bottom width of the bottom portion, such that the top width is within the range of 3.5 centimeters to 4.5 centimeters and the bottom width is within the range of 5.5 centimeters to 6.5 centimeters; and
each of the plurality of extension members has a length within a range of 3.5 centimeters to 6.5 centimeters extending outward from the bottom portion and each of the plurality of extension members has a width within a range of 0.35 centimeters to 1.5 centimeters.

17. The biological vaginal graft of claim 15, wherein the tissue graft material is composed of decellularized dermal allograft.

18. The biological vaginal graft of claim 15, wherein the tissue graft material is composed of animal-based origin.

19. The biological vaginal graft of claim 15 available for surgical attachment to the patient using the grafting medical procedure based on the existing central body, the plurality of extension members, and the apical arms without requiring modification of the vaginal graft within a surgical theater.
